(19) United States
(12) Patent Application Publication
(10) Pub. No.: US 2023/0154574 A1
(43) Pub. Date: May 18, 2023

(54) METHODS AND SYSTEMS FOR AUTOMATICALLY GENERATING CRITERIA FOR CLINICAL TRIALS

(71) Applicant: Microsoft Technology Licensing, LLC, Redmond, WA (US)

(72) Inventors: Nut LIMSOPATHAM, Bellevue, WA (US); Liang DU, Redmond, WA (US); Robin ABRAHAM, Redmond, WA (US)

(21) Appl. No.: 17/525,432

(22) Filed: Nov. 12, 2021

(51) Int. Cl. G16H 10/20; G16H 15/00; G16H 50/20; G06K 9/00

(52) U.S. Cl. CPC G16H 10/20; G16H 15/00; G16H 50/20; G06K 9/00456

(57) ABSTRACT

The methods and systems may automatically generate criteria for the different sections of the protocol document for a clinical study. The methods and systems use machine learning models to identify medical articles that are associated with the clinical study of a protocol document. The machine learning models analyze the medical articles and generate recommended criteria for the different sections of the protocol document based on the analysis.

### BACKGROUND

A clinical trial or clinical study is an important process in drug development, where the developed medicine is tested in controlled groups. If the test is successful, the medicine will later be released to the market. A protocol document (or clinical study document) is a document providing detailed information explaining how to conduct a clinical study. A clinical study is an expensive process, as the clinical study includes finding and recruiting patients and practitioners, and/or preparing physical labs, medicines, or chemicals for testing with the groups of patients. A failure during the study requires the rewriting of the protocol document and repeating the whole trial process according to the new protocol. Therefore, developing the protocol document is a crucial step for a clinical trial or clinical study.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Some implementations relate to a method for generating criteria for a clinical study. The method may include receiving a protocol document for a clinical study for a medical condition, wherein the protocol document includes one or more sections that provide different information for the clinical study. The method may include receiving a plurality of medical articles that are related to the clinical study of the protocol document. The method may include processing text from each medical article of the plurality of medical articles using at least one machine learning model. The method may include identifying criteria for the one or more sections of the protocol document based on processing each medical article by the at least one machine learning model. The method may include generating recommended criteria for the one or more sections of the protocol document by aggregating the criteria from each medical article. The method may include outputting the recommended criteria for the one or more sections of the protocol document.

Some implementations relate to a method for generating eligibility criteria for a clinical study. The method may include receiving a plurality of medical articles related to a clinical study. The method may include processing text from the plurality of medical articles using at least one machine learning model. The method may include identifying one or more portions of the text that discusses selection criteria for selecting individuals to participate in the clinical study. The method may include generating recommended eligibility criteria for the clinical study based on the selection criteria from the medical article. The method may include outputting the recommended eligibility criteria for the clinical study.

Some implementations relate to a system. The system may include a memory to store data and instructions; and at least one processor operable to communicate with the memory, wherein the at least one processor is operable to: receive a protocol document for a clinical study for a medical condition, wherein the protocol document includes one or more sections that provide different information for the clinical study; receive a plurality of medical articles that are related to the clinical study of the protocol document; process text from each medical article of the plurality of medical articles using at least one machine learning model; identify criteria for the one or more sections of the protocol document based on the processing by the at least one machine learning model of each medical article; generate recommended criteria for the one or more sections of the protocol document by aggregating the criteria from each medical article; and output the recommended criteria for the one or more sections of the protocol document.

Additional features and advantages will be set forth in the description that follows. Features and advantages of the disclosure may be realized and obtained by means of the systems and methods that are particularly pointed out in the appended claims. Features of the present disclosure will become more fully apparent from the following description and appended claims, or may be learned by the practice of the disclosed subject matter as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. Understanding that the drawings depict some example embodiments, the embodiments will be described and explained with additional specificity and detail through the use of the accompanying drawings in which:

FIG. 1 illustrates an example environment for generating criteria for a clinical study in accordance with implementations of the present disclosure.

FIG. 2 illustrates example text from a medical article in accordance with implementations of the present disclosure.

FIG. 3 illustrates an example graphical user interface (GUI) displaying recommended eligibility criteria for a clinical study in accordance with implementations of the present disclosure.

FIG. 4 illustrates an example method for generating criteria for a clinical study in accordance with implementations of the present disclosure.

FIG. 5 illustrates an example method for generating eligibility criteria for a clinical study in accordance with implementations of the present disclosure.

### DETAILED DESCRIPTION

A clinical trial or clinical study is an important process in drug development, where the developed medicine is tested in controlled groups. A protocol document (or clinical study document) is a document providing detailed information for how to conduct a clinical study. For example, the protocol document includes information, such as, study title, study description, study design, steps of the procedure, eligibility criteria, and/or how the results are measured. The protocol document is written before the clinical study is started and individuals are recruited to participate in the clinical study. Normally, a protocol document is written and reviewed by a committee of experts to determine whether the protocol document is in good shape to execute.

The clinical study is an expensive process, as the clinical study includes finding relevant individuals for a study group for the clinical study, recruiting the individuals and practitioners for the study group, and preparing physical labs and medicines, or chemicals, for testing with the study group. If a failure occurs during the clinical study, the protocol document is rewritten and the whole trial process is repeated according to the new protocol.

One main section of the protocol document includes defining cohort selection criteria, also referred to as eligibility criteria, which contains inclusion and exclusion criteria for selecting individuals (e.g., patients or subjects) to participate in a study group for the clinical study. The inclusion criteria provides features or characteristics of individuals to include in the study group for the clinical study (e.g., include patients with an age between 20 years old and 80 years). The exclusion criteria provides features or characteristics of individuals to exclude from the study group for the clinical study (e.g., exclude women or exclude pregnant women).

The eligibility criteria section of the protocol document is typically defined and reviewed by a committee of clinical study experts. In many cases, an error occurs in the eligibility criteria (e.g., eligibility criteria that is too specific may result in being unable to recruit enough individuals for the study group), resulting in the rewriting of the protocol document and repeating the whole trial process.

The present disclosure provides methods and systems for automatically generating criteria for clinical trials, and thus, reducing the failures of the clinical trials, which may costs multiple million dollars to the pharmaceutical industry. The present disclosure identifies medical articles related to the clinical study and uses machine learning models to analyze the medical articles to determine recommended criteria for the different sections of a protocol document for the clinical trials. As such, the present disclosure includes several practical applications that provide benefits and/or solve problems associated with developing the criteria for clinical trials.

The present disclosure uses a deep neural network-based language generation machine learning model to encode insights gained from the medical articles. The machine learning model may be based on sequence-to-sequence transformer networks. In one example use case, the machine learning model automatically generates the eligibility criteria section of the protocol document for a clinical study based on the analysis of the medical articles. The machine learning model takes textual information of the medical articles and extracts the eligibility criteria information. In addition, the machine learning model processes the text of the medical articles to understand the outcome of the research or study. The machine learning models uses the eligibility criteria information and the outcome of the research or the studies to determine an eligibility criteria for the clinical study. The machine learning model may aggregate the eligibility criteria determined from each medical article processed and output a recommended eligiblity criteria for the protocol document based on the analysis.

The machine learning model may output the recommended criteria for the different sections of the protocol document and present the recommended criteria in a list on a display. In addition, the protocol document may be automatically updated with the recommended criteria.

As such, the present disclosure may improve the development of the criteria for a clinical study by using a deep learning system that leverages the recent medical publications to automatically determine criteria for different sections of the protocol document.

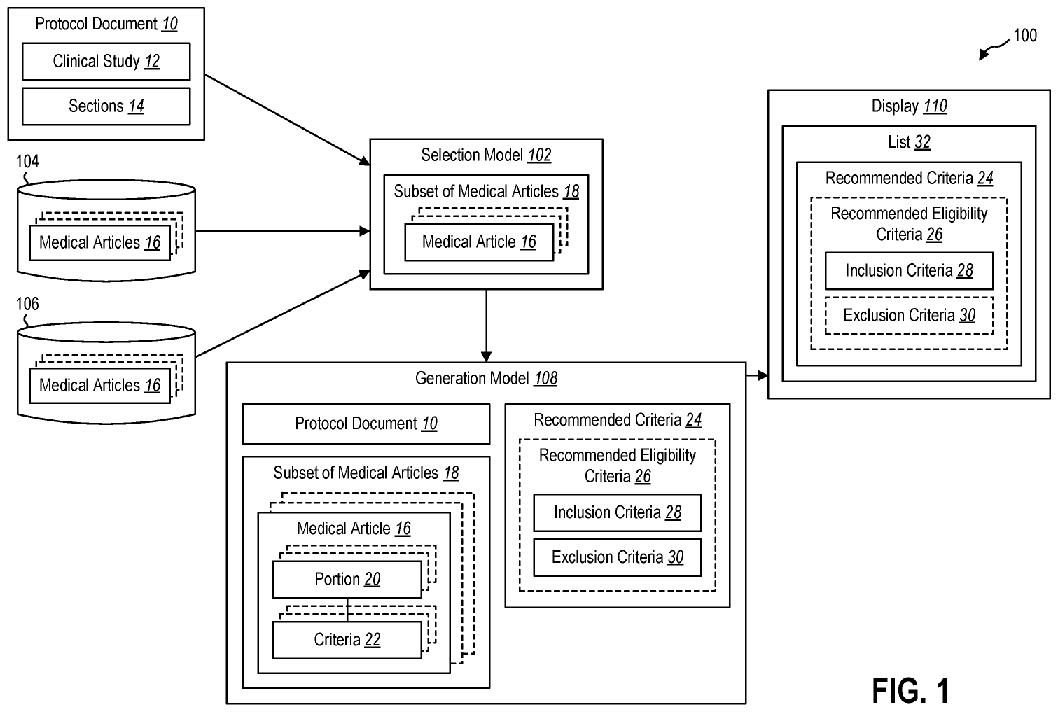

Referring now to FIG. 1, illustrated is an example environment 100 for use with automatically generating criteria for different sections 14 of a protocol document 10 for a clinical study 12. The clinical study 12 is an important process in medicine development, where the developed medicine is tested in controlled groups of individuals or patients (e.g., cohorts of patients). If the clinical study is successful, the developed medicine may be released to the market. A protocol document 10 provides detailed information for conducting the clinical study 12. The protocol documents 10 may include a plurality of sections 14, where each section 14 provides different information for the clinical study 12. For example, the sections 14 may include, but are not limited to, study title, study description, study design, steps of the procedure, eligibility criteria of individuals for the study, outcome measures, and/or how the results are measured for the study.

The environment 100 may include a selection model 102 that receives the protocol document 10 and identifies the clinical study 12 of the protocol document 10. The selection model 102 may access one or more datastores 104, 106 up to n datastores (where n is a positive integer) with a plurality of medical articles 16. The plurality of medical articles 16 may include, but are not limited to, journal articles, conference papers, medical reports, internal reports from research teams, experimental reports from companies, and/or any other documents (published or internal company documents) that may discuss medical issues. One example datastore 104, 106 includes PubMed.

The plurality of medical articles 16 may be stored by medical condition and/or disease. In addition, the plurality of medical articles 16 may be stored by different content providers. For example, the datastore 104 may include public medical articles 16 published by universities or research groups and the datastore 104 may include internal medical articles from a company (internal reports or studies performed by the company).

The selection model 102 may identify the medical articles 16 that are related to the clinical study 12 or a topic of the protocol document 10. The related medical articles may include any medical articles 16 that are in the topics or related to the topics that the clinical study 12 focuses on. Examples of related medical articles 16 include medical articles discussing the same or similar diseases or conditions as included in the protocol document 10 and/or medical articles with the same or similar study description as included in the protocol document 10. If the selection model 102 determines that the medical article 16 is related to the clinical study 12 and/or the topics of the protocol document 10, the selection model 102 adds the medical article 16 to a subset of the medical articles 18 that are related to the clinical study 12 and/or a topic of the protocol document 10. For example, if a protocol document 10 is about a study of “drug A” in preventing COVID-19. The selection model 102 may identify a medical article 16 reporting effects of a chemical in “Drug A” significantly impacting pregnant women as a related medical article to the clinical study 12 and the selection model 102 may add the medical article 16 to a subset of the medical articles 18. If the selection model 102 determines that the medical article 16 is not related to the clinical study 12 and/or the topics of the protocol document 10, the selection model 102 does not include the medical article 16 into the subset of the medical articles 18.

In some implementations, the selection model 102 sets a timeframe and only obtains the medical articles 16 within the timeframe from the datastores 104, 106. One example timeframe includes the past three years. Thus, the selection model 102 may only obtain the medical articles 16 published or written within the past three years to analyze and determine which medical articles 16 to include in the subset of the medical articles 18.

Another example includes a timeframe based on a previous output for the protocol document 10. If the protocol document 10 is being updated (e.g., updating the eligibility criteria section) or if a different clinical study 12 is being performed for the same vaccine included in a protocol document from a year ago, the selection model 102 may set the timeframe based on the previous output for the protocol document 10 (e.g., last year). As such, instead of reviewing all the medical articles 16 again, the selection model 108 may limit the timeframe for the medical articles 16 to obtain from the datastores 104, 106 to the newly published or newly written medical articles 16 (e.g., published or written after a year ago).

The generation model 108 may receive the subset of the medical articles 18 and may analyze the text of the medical articles 16 included in the subset of the medical articles 18 using one or more natural language processing (NLP) machine learning models. In some implementations, the generation model 108 uses sequence-to-sequence transformer models to analyze the text of the subset of the medical articles 18.

The machine learning models of the generation model 108 may be trained using a database of protocol documents that cover a variety of medical conditions and/or diseases used for previous clinical studies as the training input. The training input may include several hundred thousand of past protocol documents for previously performed clinical studies. In addition, the training input may include several hundred thousand medical articles that cover a variety of medical conditions or diseases. Alternatively, the generation model 108 may be based on a powerful pre-trained language model, such as, a generative pre-trained transformer (GPT)-3, which is trained on billions of documents. Using zero-shot or few-shot learning, the GPT-3 generation model may generate the output from the knowledge and/or insights extracted from the medical articles.

The machine learning models may be trained to identify portions 20 of the medical articles related to the different sections 14 of the protocol documents 10. As such, the corpus used for the training input of the machine learning models may include a large volume of existing protocol documents for past clinical studies performed and a large volume of different medical articles.

The generation model 108 may analyze each medical article 16 of the subset of the medical articles 18 separately. For each medical article 16, the generation model 108 identifies different portions 20 of the medical article 16 that are related to the sections 14 of the protocol document 10 and extracts the information from the medical article 16. The generation model 108 also analyzes the text of the medical article 16 and determines what type of research or study the medical article discusses and determines a result of the research or study based on the analysis of the text.

Different medical articles 16 may have different portions 20 that may be related to the different sections 14 of the protocol document 10. As such, in some implementations, the generation model 108 only identifies portions 20 of the medical article 16 related to a specific section 14 of the protocol document 10 during the analysis of the medical article 16. In one example, the generation model 108 identifies a portion 20 of the medical article 16 related to the eligibility criteria section 14 of the protocol document and extracts information relating to the eligibility criteria section 14. In another example, the generation model 108 identifies a portion 20 of the medical article 16 related to the outcome measures section 14 of the protocol document 10 and extracts information relating to outcome measures from the medical article 16.

The generation model 108 may generate criteria 22 for the one or more sections 14 of the protocol document 10 based on the insights gained from the medical article 16. The insights may be based on new work published in the medical articles 16. The insights may be gained by the generation model 108 processing the information extracted from the identified portions 20 of the medical article 16. For example, the generation model 108 extracts from a medical article the following medical insight “based on our extensive study, the chemical X has significant impacts on restricting early stage chromosome growth and development.”

The generation model 108 may generate recommended criteria 24 for the one or more sections 14 of the protocol document 10 by aggregating the criteria 22 identified for each medical article 16 in the subset of the medical articles 18. The aggregation may be based on the uniqueness and/or distinction of individual criteria 22 from the different medical articles 16 and/or the existing criteria in the target protocol document 10. For example, if two medical articles both recommend inclusion criteria “male,” the generation model 108 may keep one of the “male” inclusion criteria to avoid duplication.

In some implementations, the recommended criteria 24 includes recommended eligibility criteria 26 for the clinical study 12 of the protocol document 10. The recommended eligibility criteria 26 includes inclusion criteria 28 that provides features or characteristics of individuals to recruit for participating in the study group for the clinical study 12. An example inclusion criteria 28 is include patients with an age between 20 years old and 80 years old in the study group for the clinical study 12. The exclusion criteria 30 provides features or characteristics of individuals to exclude from participating in the study group for the clinical study 12. An example exclusion criteria 30 is exclude pregnant women from the study group for the clinical study 12. The recommended eligibility criteria 26 may be based on the insights extracted by the generation model 108. For example, the generation model 108 extracts the following medical insight from a medical article “based on our extensive study, the chemical X has significant impacts on restricting early stage chromosome growth and development.” From the extracted insight, the generation model 108 may generate an exclusion criteria 30 for “women in early pregnancy,” as the chemical X could have a strong effect in baby growth. In some implementations, the recommended criteria 24 includes recommended criteria for outcome measures or the study design.

The generation model 108 may output the recommended criteria 24 to a display 110 of a device. The recommended criteria 24 may be presented on the display 110, for example, in a list 32. The list 32 may include the title of the clinical study 12 and the section 14 of the protocol document 10 for the recommended criteria 24 as a heading in the list 32. The recommended criteria 24 may be presented under the heading.

In some implementations, the recommended criteria 24 is automatically presented in the protocol document 10. The recommended criteria 24 may be visually distinct from other portions of the protocol document 10 so that the recommended criteria 24 is easily identified in the protocol document 10 (e.g., highlighted in a different color).

In some implementations, a user of the device may review the recommended criteria 24. For example, a clinical study expert may review the recommended criteria 24 and approve the recommended criteria 24 before the recommended criteria 24 is implemented in the clinical study 12. In addition, a clinical study expert may make modifications or improvements to the recommend criteria 24 before the recommended criteria 24 is implemented in the clinical study 12.

The environment 100 may have multiple machine learning systems and/or machine learning models running simultaneously. For example, the selection model 102 and/or the generation model 108 may use one or more machine learning models for natural language processing (NLP) to analyze the text of the protocol document 10 and/or the text of the medical articles 16. For example, the machine learning models may be transformer networks, such as, but not limited to Bidirectional Encoder Representations from Transformers (BERT) models and/or Generative Pre-Trained Transformers (GPT). The transformer networks may be trained by processing the raw data of text from the protocol documents and/or the medical articles. Other examples of the machine learning models may include, but are not limited to, Embeddings from Language Models (ELMO), and/or any other machine learning model for NLP.

In some implementations, one or more computing devices (e.g., servers and/or devices) are used to perform the processing of environment 100. The one or more computing devices may include, but are not limited to, server devices, personal computers, a mobile device, such as, a mobile telephone, a smartphone, a PDA, a tablet, or a laptop, and/or a non-mobile device. The features and functionalities discussed herein in connection with the various systems may be implemented on one computing device or across multiple computing devices. For example, the selection model 102, the datastores 104, 106, the generation model 108, and/or the display 110 are implemented wholly on the same computing device. Another example includes one or more subcomponents of the selection model 102, the datastores 104, 106, the generation model 108, and/or the display 110 implemented across multiple computing devices. Moreover, in some implementations, the selection model 102, the datastores 104, 106, the generation model 108, and/or the display 110 may be implemented are processed on different server devices of the same or different cloud computing networks.

In some implementations, each of the components of the environment 100 is in communication with each other using any suitable communication technologies. In addition, while the components of the environment 100 are shown to be separate, any of the components or subcomponents may be combined into fewer components, such as into a single component, or divided into more components as may serve a particular embodiment. In some implementations, the components of the environment 100 include hardware, software, or both. For example, the components of the environment 100 may include one or more instructions stored on a computer-readable storage medium and executable by processors of one or more computing devices. When executed by the one or more processors, the computer-executable instructions of one or more computing devices can perform one or more methods described herein. In some implementations, the components of the environment 100 include hardware, such as a special purpose processing device to perform a certain function or group of functions. In some implementations, the components of the environment 100 include a combination of computer-executable instructions and hardware.

The environment 100 may leverages the large volume of information provided in medical publications 16 to determine the criteria 24 for different sections 14 of the protocol document 10. As such, the environment 100 may be used to automatically generate or develop the criteria for the clinical study 12 to improve the coverage of the clinical study 12 so that the clinical study 12 is more successful.

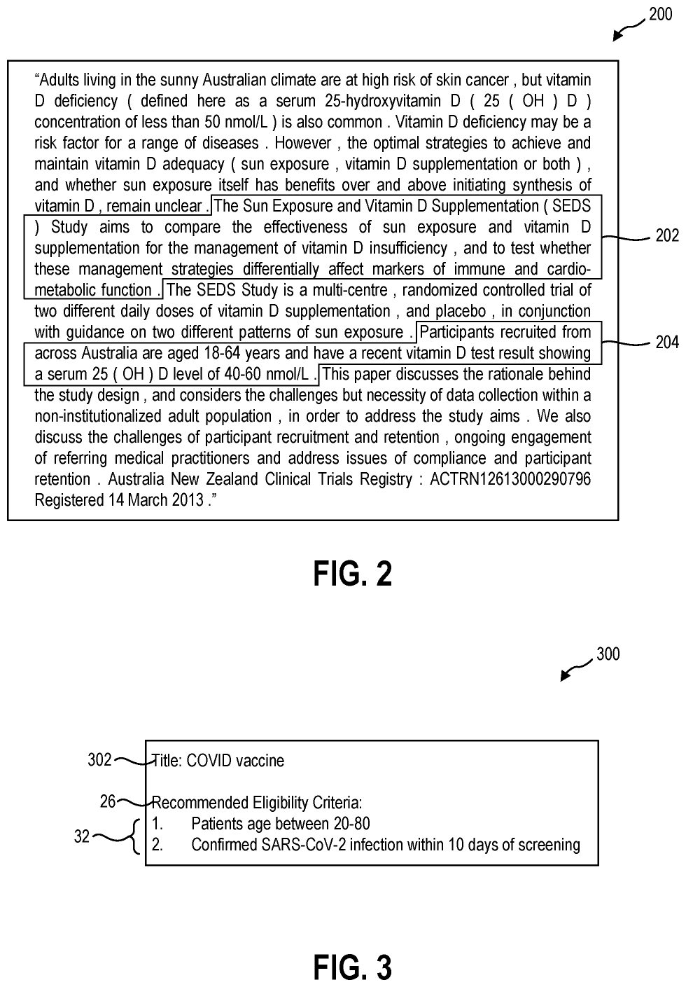

Referring now to FIG. 2, illustrated is an example text 200 from a medical article 16 (FIG. 1) processed by the selection model 102 (FIG. 1) and/or the generation model 108 (FIG. 1). The selection model 108 may process the text 200 of the medical article 16 and perform natural language processing on the text 200 and may identify a portion 202 of the text 200 that discusses the purpose of the study discussed in the medical article 16 based on processing the text 200. For example, the portion 202 of the text 200 indicates that the purpose of the study is comparing the effectiveness of sun exposure and vitamin D supplementation for the management of vitamin D.

The selection model 108 may compare the portion 202 of the text 200 with the purpose of the study to the clinical study 12 (FIG. 1) and/or topics discussed in the protocol document 10 (FIG. 1). If the selection model 108 determines that the portion 202 of the text 200 is related to the clinical study 12 and/or the topics discussed in the protocol document 10, the selection model 108 may include the medical article 16 in a subset of medical articles 18 (FIG. 1) to provide to the generation model 108 for further processing.

The generation model 108 may also process the text 200 of the medical article 16 and may perform natural language processing on the text 200. The generation model 108 may identify a portion 204 of the text 200 that discusses selection criteria for individuals to participate in the study discussed in the medical article 16. For example, the portion 204 of the text 200 discusses that participants between the ages of 18-64 that have recent vitamin D test results showing a serum 25 (OH) D level of 40-60 nmol/L were included in the study.

For example, if the clinical study 12 is for a new vitamin D supplement, the generation model 108 may gain insights from the portion 204 of the text 200 to generate the recommended eligibility criteria 26 (FIG. 1) for the participants for the clinical study 12. The generation model 108 may generate inclusion criteria 28 (FIG. 1) for the recommended eligibility criteria 26 from the insights. The inclusion criteria 28 may include participants between the ages of 18-64. The inclusion criteria 28 may also include participants with vitamin D test results showing a serum 25 (OH) D level of 40-60 nmol/L. The generation model 108 may also generate exclusion criteria 30 (FIG. 1) for the recommended eligibility criteria 26 from the insights. The exclusion criteria 30 may include do not recruit participants over 64 years of age for participating in the clinical study 12. Using the information from the study discussed in the medical article 16 may help improve the coverage of the study group for the clinical study 12.

While the above use case focused on the eligibility criteria section 14 of the protocol document 10, the generation model 108 may identify different portions of the text 200 that discussed other sections of the protocol document 10 (e.g., the study design or outcome measures) and may generate recommended criteria 24 (FIG. 1) based on the analysis of the other portions of the text 200. Moreover, the generation model 108 may process the text of a plurality of medical articles, as discussed above in reference to the text 200, when generating the recommended eligibility criteria 26.

Referring now to FIG. 3, illustrated is an example graphical user interface (GUI) 300 for displaying the recommended eligibility criteria 26 output by the generation model 108 (FIG. 1) based on the analysis of the subset of medical articles 18 (FIG. 1). The GUI 300 may be displayed on the display 110 of a device in communication with the generation model 108.

The GUI 300 may include the title 302 of the clinical study 12 (e.g., Coronavirus disease (COVID) vaccine). The GUI 300 may also include the recommended eligibility criteria 26 presented in a list 32. The list 32 includes the two inclusion criteria 28 (FIG. 1) for the COVID vaccine clinical study 12 (e.g., include patients with an age between 20-80 and include patients with confirmed SARS-CoV-2 infection within 10 days of screening).

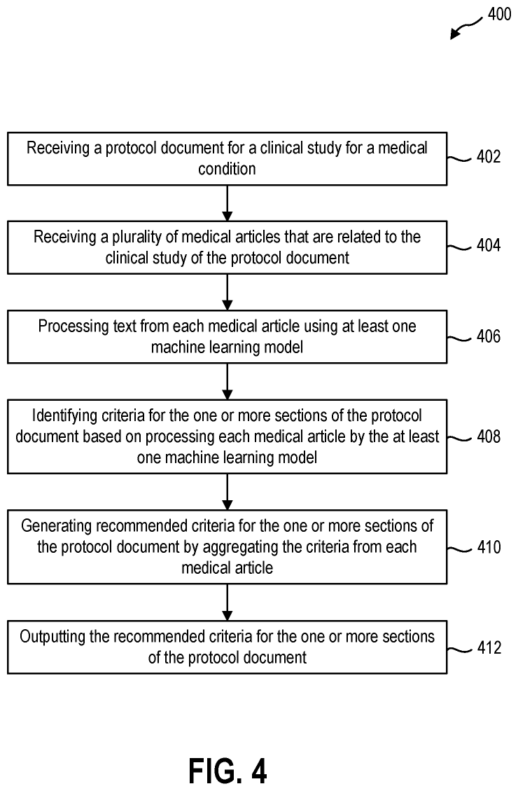

Referring now to FIG. 4, illustrated is an example method 400 for generating criteria for sections 14 (FIG. 1) of a protocol document 10 (FIG. 1). The actions of method 400 may be discussed below with reference to the architectures of FIG. 1.

At 402, the method 400 includes receiving a protocol document for a clinical study for a medical condition. The generation model 108 (FIG. 1) may receive a protocol document 10 for a clinical study 12 for a medical condition. The medical condition may be a disease and the clinical study may be for a drug or medicine for treating the disease. The protocol document 10 includes one or more sections 14 that provide different information for the clinical study 12. The one or more sections 14 of the protocol document 10 may include an eligibility criteria section for selecting individuals for a study group to participate in the clinical study 12. The one or more sections 14 of the protocol document 10 may also include an outcome measure section for the clinical study 12. As such, the protocol document 10 provides detailed information for conducting the clinical study 12 and each section 14 of the protocol document 10 provides different information for the clinical study 12.

At 404, the method 400 includes receiving a plurality of medical articles that are related to the clinical study of the protocol document. The generation model 108 may receive a subset of medical articles 18 with a plurality of medical articles 16 related to the clinical study 12 from the selection model 102. The medical articles 16 may be journal articles, conference papers, medical reports, internal reports, and/or experimental reports. Examples of related medical articles 16 include, but are not limited to, medical articles 16 discussing the same or similar diseases or conditions as included in the protocol document 10 and/or medical articles with the same or similar study description as included in the clinical study 12.

At 406, the method 400 includes processing text from each medical article of the plurality of medical articles using at least one machine learning model. The generation model 108 may analyze the text of each medical article 16 included in the subset of the medical articles 18 separately using one or more natural language processing (NLP) machine learning models. In some implementations, the generation model 108 is a transformer network model.

At 408, the method 400 includes identifying criteria for the one or more sections of the protocol document based on processing each medical article by the at least one machine learning model. For each medical article 16, the generation model 108 identifies different portions 20 of the medical article 16 that are related to the sections 14 of the protocol document 10 and extracts the information from the medical article 16. The generation model 108 also analyzes the text of the medical article 16 and determines what type of research or study the medical article discusses and determines a result of the research or study based on the analysis of the text.

Different medical articles 16 may have different portions 20 that may be related to the different sections 14 of the protocol document 10. As such, in some implementations, the generation model 108 only identifies portions 20 of the medical article 16 related to a specific section 14 of the protocol document 10 during the analysis of the medical article 16. In one example, the generation model 108 identifies a portion 20 of the medical article 16 related to the eligibility criteria section 14 of the protocol document and extracts information relating to the eligibility criteria section 14. In another example, the generation model 108 identifies a portion 20 of the medical article 16 related to the outcome measures section 14 of the protocol document 10 and extracts information relating to outcome measures from the medical article 16.

The generation model 108 may generate criteria 22 for the one or more sections 14 of the protocol document 10 based on the information obtained from the medical article 16. The information may be obtained from the portions 20 of the medical article 16 related to the sections 14 of the protocol document 10.

At 410, the method 400 includes generating recommended criteria for the one or more sections of the protocol document by aggregating the criteria from each medical article. The generation model 108 may generate recommended criteria 24 for the one or more sections 14 of the protocol document 10 by aggregating the criteria 22 identified for each medical article 16 in the subset of the medical articles 18. The recommended criteria 24 may include recommended eligibility criteria 26 with inclusion criteria 28 for the individuals to select for participating in the clinical study 12. The recommended eligibility criteria 26 may also include exclusion criteria 30 for individuals to prevent from participating in the clinical study 12. The recommended criteria 24 may also include criteria for the outcome measure section.

At 412, the method 400 includes outputting the recommended criteria for the one or more sections of the protocol document. The generation model 108 may output the recommended criteria 24 for the sections 14 of the protocol document 10. Outputting the recommended criteria 24 may include automatically updating the sections 14 of the protocol document 10 with the recommended criteria 24. Outputting the recommended criteria 24 may also include presenting the recommended criteria 24 in a list 32 on a display 110 of a device.

As such, the method 400 may be used to automatically generate or develop the criteria for different sections 14 of a protocol document 10 for the clinical study 12 to improve the coverage of the clinical study 12 so that the clinical study 12 is more successful.

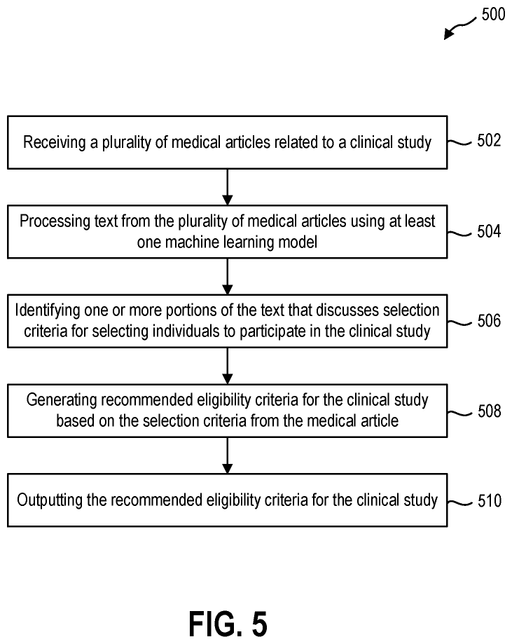

Referring now to FIG. 5, illustrated is a method 500 for generating eligibility criteria for a clinical study. The actions of method 500 may be discussed below with reference to the architectures of FIG. 1.

At 502, the method 500 includes receiving a plurality of medical articles related to a clinical study. The generation model 108 may receive a subset of medical articles 18 with a plurality of medical articles 16 related to the clinical study 12. The medical articles 16 may be journal articles, conference papers, medical reports, internal reports, and/or experimental reports. Examples of related medical articles 16 include, but are not limited to, medical articles 16 discussing the same or similar diseases or conditions as included in the protocol document 10 and/or medical articles with the same or similar study description as included in the clinical study 12.

At 504, the method 500 includes processing text from the plurality of medical articles using at least one machine learning model. The generation model 108 may process the text of the medical articles 16 and may perform natural language processing on the text.

At 506, the method 500 includes identifying one or more portions of the text that discusses selection criteria for selecting individuals to participate in the clinical study. The generation model 108 may identify one or more portions 20 of the text that discusses selection criteria for individuals to participate in the study discussed in the medical article 16.

At 508, the method 500 includes generating recommended eligibility criteria for the clinical study based on the selection criteria from the medical article. The generation model 108 may automatically generate the recommended criteria 24 based on the portions 20 of the text that discusses the selection criteria in the medical articles 16. The recommended criteria 24 may include recommended eligibility criteria 26 with inclusion criteria 28 for the individuals to select for participating in the clinical study 12. The recommended eligibility criteria 26 may also include exclusion criteria 30 for individuals to prevent from participating in the clinical study 12. The recommended eligibility criteria 26 may be generated based on an aggregation of the selection criteria 22 from different medical articles 16 of the subset of medical articles 18.

At 510, the method 500 includes outputting the recommended eligibility criteria for the clinical study. The generation model 108 outputs the recommended eligibility criteria 24 for the clinical study 12. Outputting the recommended eligibility criteria 24 may include presenting a list 32 with the inclusion criteria 28 for the individuals on a display 110. Outputting the recommended eligibility criteria 24 may also include automatically adding the recommended eligibility criteria 26 to a protocol document 10 for the clinical study 12. The protocol document 10 may be presented on a display 110 and the recommended eligibility criteria 26 may be visually distinct (e.g., a different color) from other sections 14 of the protocol document 10, making the recommended eligibility criteria 26 easy to identify.

As such, the method 500 may be used to generate or develop the eligibility criteria section 14 of clinical study 12 to improve the coverage of the study group for the clinical study 12 so that the clinical study 12 is more successful.

As illustrated in the foregoing discussion, the present disclosure utilizes a variety of terms to describe features and advantages of the model evaluation system. Additional detail is now provided regarding the meaning of such terms. For example, as used herein, a “machine learning model” refers to a computer algorithm or model (e.g., a transformer model, a classification model, a regression model, a language model, an object detection model) that can be tuned (e.g., trained) based on training input to approximate unknown functions. For example, a machine learning model may refer to a neural network (e.g., a transformer neural network, a convolutional neural network (CNN), deep neural network (DNN), recurrent neural network (RNN)), or other machine learning algorithm or architecture that learns and approximates complex functions and generates outputs based on a plurality of inputs provided to the machine learning model. As used herein, a “machine learning system” may refer to one or multiple machine learning models that cooperatively generate one or more outputs based on corresponding inputs. For example, a machine learning system may refer to any system architecture having multiple discrete machine learning components that consider different kinds of information or inputs.

The techniques described herein may be implemented in hardware, software, firmware, or any combination thereof, unless specifically described as being implemented in a specific manner. Any features described as modules, components, or the like may also be implemented together in an integrated logic device or separately as discrete but interoperable logic devices. If implemented in software, the techniques may be realized at least in part by a non-transitory processor-readable storage medium comprising instructions that, when executed by at least one processor, perform one or more of the methods described herein. The instructions may be organized into routines, programs, objects, components, data structures, etc., which may perform particular tasks and/or implement particular data types, and which may be combined or distributed as desired in various embodiments.

Computer-readable mediums may be any available media that can be accessed by a general purpose or special purpose computer system. Computer-readable mediums that store computer-executable instructions are non-transitory computer-readable storage media (devices). Computer-readable mediums that carry computer-executable instructions are transmission media. Thus, by way of example, and not limitation, embodiments of the disclosure can comprise at least two distinctly different kinds of computer-readable mediums: non-transitory computer-readable storage media (devices) and transmission media.

As used herein, non-transitory computer-readable storage mediums (devices) may include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

The steps and/or actions of the methods described herein may be interchanged with one another without departing from the scope of the claims. Unless a specific order of steps or actions is required for proper operation of the method that is being described, the order and/or use of specific steps and/or actions may be modified without departing from the scope of the claims.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one implementation” or “an implementation” of the present disclosure are not intended to be interpreted as excluding the existence of additional implementations that also incorporate the recited features. For example, any element described in relation to an implementation herein may be combinable with any element of any other implementation described herein. Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to implementations disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the implementations that falls within the meaning and scope of the claims is to be embraced by the claims.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for generating criteria for a clinical study, comprising:
receiving a protocol document for a clinical study for a medical condition, wherein the protocol document includes one or more sections that provide different information for the clinical study;
receiving a plurality of medical articles that are related to the clinical study of the protocol document;
processing text from each medical article of the plurality of medical articles using at least one machine learning model;
identifying criteria for the one or more sections of the protocol document based on processing each medical article by the at least one machine learning model;
generating recommended criteria for the one or more sections of the protocol document by aggregating the criteria from each medical article; and
outputting the recommended criteria for the one or more sections of the protocol document.

2. The method of claim 1, wherein the one or more sections of the protocol document include an eligibility criteria section for selecting individuals for a study group to participate in the clinical study.

3. The method of claim 2, wherein the recommended criteria includes recommended eligibility criteria for the eligibility criteria section of the protocol document.

4. The method of claim 3, wherein the recommended eligibility criteria includes inclusion criteria for the individuals and exclusion criteria for the individuals.

5. The method of claim 1, wherein the one or more sections include an outcome measure section for the clinical study and the recommended criteria includes criteria for the outcome measure section.

6. The method of claim 1, wherein the plurality of medical articles include one or more of journal articles, conference papers, medical reports, internal reports, or experimental reports.

7. The method of claim 1, further comprising:
automatically updating the one or more sections of the protocol document with the recommended criteria.

8. The method of claim 1, wherein outputting the recommended criteria includes presenting the recommended criteria for each of the one or more sections in a list

9. The method of claim 1, wherein the medical condition is a disease and the clinical study is for a drug or medicine for treating the disease.

10. The method of claim 1, wherein the output is presented on a display.

11. The method of claim 1, wherein the at least one machine learning model is a transformer network.

12. A method for generating eligibility criteria for a clinical study, comprising:
receiving a plurality of medical articles related to a clinical study;
processing text from the plurality of medical articles using at least one machine learning model;
identifying one or more portions of the text that discusses selection criteria for selecting individuals to participate in the clinical study;
generating recommended eligibility criteria for the clinical study based on the selection criteria from the medical article; and
outputting the recommended eligibility criteria for the clinical study.

13. The method of claim 12, wherein the recommended eligibility criteria includes inclusion criteria for the individuals.

14. The method of claim 12, wherein the recommended eligibility criteria includes exclusion criteria for the individuals.

15. The method of claim 12, wherein outputting the recommended eligibility criteria includes presenting a list with inclusion criteria for the individuals on a display.

16. The method of claim 12, wherein outputting the recommended eligibility criteria includes automatically adding the recommended eligibility criteria to a protocol document for the clinical study; and
presenting the protocol document with the recommended eligibility criteria on a display.

17. The method of claim 16, wherein the recommended eligibility criteria is visually distinct from other sections of the protocol document.

18. The method of claim 12, wherein the at least one machine learning model is a transformer network.

19. The method of claim 18, wherein the recommended eligibility criteria is generated based on an aggregation of the selection criteria from different medical articles of the plurality of medical articles.

20. A system comprising,
a memory to store data and instructions; and
at least one processor operable to communicate with the memory, wherein the at least one processor is operable to:
receive a protocol document for a clinical study for a medical condition, wherein the protocol document includes one or more sections that provide different information for the clinical study;
receive a plurality of medical articles that are related to the clinical study of the protocol document;
process text from each medical article of the plurality of medical articles using at least one machine learning model;
identify criteria for the one or more sections of the protocol document based on the processing by the at least one machine learning model of each medical article;
generate recommended criteria for the one or more sections of the protocol document by aggregating the criteria from each medical article; and
output the recommended criteria for the one or more sections of the protocol document.
